SEQUENCE LISTING (2 sequences)

SEQ ID NO: 1 - protein, 5 aa, Homo sapiens and Bos taurus
  MOD_RES (1)...(3): PHOSPHORYLATION of Ser
SSSEE

SEQ ID NO: 2 - protein, 5 aa, Unknown
  Mineral Target Binding Sequence
XSSEE

(19) United States
(12) Patent Application Publication
(10) Pub. No.: US 2003/0096741 A1
(43) Pub. Date: May 22, 2003

(54) Enhanced mineral delivery

(76) Inventors: Charles W. Slattery, Yucaipa, CA (US); John Leonora, Yucaipa, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/885,593

(22) Filed: Jun. 19, 2001

Related U.S. Application Data
(60) Provisional application No. 60/214,606, filed on Jun. 27, 2000.

(51) Int. Cl. A61K 38/17

(52) U.S. Cl. 514/12; 514/167; 530/360; 424/617; 514/478

(57) ABSTRACT

Recombinant beta-casein (β-CN) proteins and peptides which include the mineral-binding target sequence Xaa-SerP-SerP-Glu-Glu, where SerP is phosphoserine, in which Xaa is any amino acid other than serine or phosphoserine. These phosphopeptides are complexed with minerals, such as calcium, and used as dietary supplements.

### BACKGROUND OF THE INVENTION

1. Field of the Invention

The present invention relates to recombinant or synthetic beta-casein, phosphopeptides derived therefrom, which include the sequence SerP-SerP-Glu-Glu. These peptides can be loaded with metal ions such as calcium, and administered to an individual as a dietary supplement. The present invention also contemplates nutritional compositions containing those materials.

2. Description of the Related Art

It is well known that various minerals, either in large amounts or as trace elements, are important for the growth and maintenance of various body systems. These minerals include calcium and phosphate for the building and maintenance of bones and teeth, as well as magnesium, iron, zinc, copper, selenium, manganese, molybdenum and chromium. All of these minerals must be supplied in the diet. Of particular interest in recent years has been the availability of calcium, which is important during bone growth in infancy and childhood, bone healing after injury, and in the prevention of osteoporosis, particularly afflicting women, but also men as they age. With the emphasis on calcium in the diet have come recommendations regarding the use of milk products and/or calcium supplements throughout life to build up bone mass and maintain it as the body ages.

Bone also contains phosphate and protein, mostly collagen. Most calcium supplements do not provide these components, which are also necessary for skeletal growth and maintenance. Calcium bioavailability, as well as bioavailability of many other minerals, is greater in milk than in diets containing other proteins and an equivalent amount of calcium, a fact that has been attributed to casein (CN) proteins and the casein phosphopeptides (CPP) derived from CN (Lee, et al., Br. J. Nutr. 49:67 (1983)). The difference was attributed to the ability of CN and the CPP to bind both calcium and inorganic phosphate which makes the combination more soluble and available for absorption rather than insoluble and excreted in the feces (Kitts, et al., Trends Food Sci. Technol 3:31 (1992)). Subsequently, bovine CPP were isolated, complexed with calcium and inorganic phosphate and found to be active in increasing calcium absorption (Sato, et al., J. Nutr. Sci. Vitaminol. Tokyo 32:67 (1989)). These same CPP complexes, when applied to teeth, resulted in a decrease in tooth demineralization and the formation of caries in rats (Reynolds, et al., J. Dent. Res. 74:1272 (1995)).

One of the problems associated with the use of cow's milk as a source of calcium and phosphate arises from the inability of many people to digest lactose. Other problems come from allergies to milk proteins and peptides derived therefrom by hydrolysis. Even though small in size with diminished allergenicity, CPP derived from tryptic hydrolysis of bovine β-CN can still generate some allergic response which may affect sensitive individuals (Heddleson, et al., J. Dairy Sci. 80:1971 (1976)). There may also be a problem with calcium being bound so tightly that it is not bioavailable (Kitts et al, supra). There are also problems with calcium supplementation in the form of calcium carbonate or calcium citrate. For example, calcium carbonate has been reported to be ineffective in preventing the decline in spinal mass in early postmenopausal women (Ettinger, et al., Ann. Int. Med. 106:40 (1987)). Calcium citrate is apparently better utilized (C. Pak, Nat'l Kidney Found. CRN Quarterly 12:8 (1988)), but the citrate also may enter the blood and prevent complete calcium utilization. Furthermore, taking too much calcium as either the carbonate or the citrate salt in order to overcome these absorption defects can lead to other problems. For example, excess calcium in the diet can lead to reduced absorption of zinc, another essential mineral (Wood, et al., Am. J. Clin. Nutr. 65: 1803 (1997)). These problems suggest the need for an improved method for delivery of calcium phosphate and other minerals.

The major calcium binding amino acid sequence in both human and bovine β-CN is SerP-SerP-SerP-Glu-Glu (SEQ ID NO: 1) (Bonsing, et al., Aust. J. Biol Sci. 41:527 (1988); Lonnerdal, et al., FEBS Letters 269:153 (1990)). This sequence is also found in sheep, goat, rabbit and pig β-CN. This sequence differs somewhat from the mouse and rat β-CN, where it appears that there are only two SerP in each of the major calcium binding sequences. β-CN is the major calcium-binding protein in human milk, where there is essentially no α-CN fraction (Groves, et al., Arch. Biochem. Biophys. 140:47 (1970)). β-CN is variably phosphorylated at serine (Ser) residues, ranging from zero up to five phosphorylated Ser residues (Groves, et al., supra). The variant with four SerP residues (β-CN-4P), having all three Ser residues in the target sequence phosphorylated, and that with two SerP residues (β-CN-2P), having only two of the three Ser residues in the target sequence phosphorylated, are present in almost equal amounts, each constituting nearly 30% of the total (Sood, et al., Arch. Biochem. Biophys. 242:355 (1985)). Calcium binding studies of the five different variants gave results which were used to calculate the dissociation constants shown in Table 1.

TABLE 1

| Human | Average dissociation | |
|---|---|---|
| β-CN variant | Constant | Reference |
| 1P | 11.7 × 10⁻⁴ M | Sood et al., J. Dairy Sci. |
| | | 77:405, 1994 |
| 2P | 12.1 × 10⁻⁴ M | Sood et al., J. Dairy Sci. |
| | | 75:2937, 1992 |
| 3P | 8.6 × 10⁻⁴ M | Sood et al., Arch. Biochem. |
| | | Biophys. 277:415, 1990* |
| 4P | 8.1 × 10⁻⁴ M | Sood et al., J. Dairy Sci. |
| | | 80:1554, 1997 |
| 5P | 4.2 × 10⁻⁴ M | Sood et al., Arch. Biochem. |
| | | Biophys. 242:355, 1985 |

*Value derived from Scatchard Plot of binding data. See G. Scatchard, Ann. New York Acad. Sci. 51, 660 (1949).

These values may be compared to the bovine β-CN calcium dissociation constant of 6.5×10⁻⁴ M. The data in Table 1 indicate the 5P variant would have the strongest calcium binding, indicated by its low dissociation constant.(Slattery et al., Biophys. Chem. 1:104 (1973)). For human β-CN variants that are phosphorylated at 4 or fewer sites (i.e., having a dissociation constant of 8.1×10⁻⁴ M or less), calcium is released more easily than from bovine β-CN.

Reynolds (WO 98/40406) has shown that the 3P variant having the sequence motif SerP-SerP-SerP-Glu-Glu (SEQ ID NO: 1) showed calcium and phosphate binding 2.5 times as strong as that of 2P synthetic homologues having the motif SerP-SerP-Glu-Glu. The present inventors have recognized that one problem with previous calcium and other mineral supplements having strong ion binding is that such compositions do not release these ions easily. This problem has apparently not been recognized in the prior art. Thus, there is a need for mineral supplements with excellent bioavailability to target tissues. The present invention addresses this need.

### SUMMARY OF THE INVENTION

In one embodiment, the invention includes an isolated recombinant or synthetic beta-casein (β-CN) variant or a peptide, comprising the amino acid sequence Xaa-SerP-SerP-Glu-Glu, wherein Xaa is an amino acid other than serine or phosphoserine. It also includes a composition comprising an isolated, preferably non-murine beta-casein (β-CN) variant or a peptide, comprising the amino acid sequence Xaa-SerP-SerP-Glu-Glu, wherein Xaa is an amino acid other than serine or phosphoserine, in combination with a mineral. Preferably the β-CN is recombinant, and in any of the embodiments of the invention, the mineral is preferably selected from inorganic phosphate, calcium, chromium, magnesium, iron, zinc, copper, selenium, manganese, and molybdenum. The compositions of the invention may further comprise a carbamoyl compound, such as carbamoyl phosphate or carbamoyl aspartate, and may also advantageously include Vitamin D or 1,25-Dihydroxy Vitamin D (1,25-Dihydroxycholecalciferol), or a combination of Vitamin D and 1,25-Dihydroxy Vitamin D.

The present invention also includes a method for delivering a mineral to an individual in need thereof, comprising administering a beta-casein (β-CN) variant or a peptide, comprising the amino acid sequence Xaa-SerP-SerP-Glu-Glu, wherein Xaa is an amino acid other than serine or phosphoserine, in combination with the mineral. Preferably, the mineral is selected from the group consisting of calcium, chromium, magnesium, iron, zinc, copper, selenium, manganese, molybdenum, and inorganic phosphate.

The present invention also includes a method for delivering a mineral to an individual in need thereof, comprising administering an isolated peptide having between about 8 and 50 amino acids, comprising the amino acid sequence Xaa-SerP-SerP-Glu-Glu, wherein Xaa is an amino acid other than serine, phosphoserine, aspartate, or glutamate, in combination with the mineral. The peptide may advantageously be synthetic or recombinant.

One embodiment of the invention is a dental formulation (such as a dentrifice, chewing gum or mouthwash) comprising a recombinant or synthetic beta-casein (β-CN) variant or peptide comprising the amino acid sequence Xaa-SerP-SerP-Glu-Glu, wherein Xaa is an amino acid other than serine, phosphoserine.

One important aspect of the invention is a method for increasing bone mass or preventing or reducing bone loss, comprising administering to a vertebrate a synthetic or recombinant peptide or protein comprising the amino acid sequence Xaa-SerP-SerP-Glu-Glu, wherein Xaa is an amino acid other than serine, phosphoserine, in combination with calcium. This method also may include administering a carbamoyl compound to the vertebrate, such as carbamoyl phosphate or carbamoyl aspartate, and may also optionally include administering Vitamin D or 1,25-Dihydroxy Vitamin D (1,25-Dihydroxycholecalciferol) or a combination of Vitamin D and 1,25-Dihydroxy Vitamin D.

### DETAILED DESCRIPTION OF THE INVENTION

The sequence Ser-Ser-Ser-Glu-Glu is highly conserved in non-murine beta caseins. Typically, this sequence is phosphorylated at one, two, or three of the serine residues. The present invention provides recombinant and synthetic variants of beta-casein (β-CN), or peptides or polypeptides, which include the mineral-binding target sequence Xaa-SerP-SerP-Glu-Glu (SEQ ID NO: 2), where SerP is phosphoserine, and Xaa is one or more amino acids other than serine or phosphoserine. Xaa may be selected from any of the 20 common amino acids in mammalian proteins, and more specifically any of the hydrophobic amino acids, any of the hydrophilic amino acids, any of the charged amino acids, or any of the polar amino acids. Note that this sequence includes only two, not three, serine residues in the region of interest. This modification of the native beta-casein is intended to bind calcium or other minerals, but not so tightly that the mineral stays bound to the sequence through the entire digestion process, as is possible with the native, unmodified beta-casein protein or relevant fragment thereof containing the sequence of interest.

In another embodiment of the invention, casein phosphopeptides (CPP) are provided which are produced by proteolytic digestion of said human β-CN variants with trypsin, pepsin, chymotrypsin or any other appropriate enzyme which results in production of one or more peptides that contain the amino acid sequence of SEQ ID NO: 2. Polypeptides according to the present invention that comprise multiple target binding sequences according to SEQ ID NO. 2, separated by cleavage sites for proteolytic enzymes are also contemplated. Such polypeptides would include 2, 3, 4, 5, 10 or more pentapeptide sequences according to SEQ ID NO. 2, separated by proteolytically-cleavable peptide sequence. The determination of the ability of any desired proteolytic enzyme to produce these peptides can be determined by standard methods, including digestion, high performance liquid chromatography (HPLC) of the digest, followed by amino acid sequencing.

A preferred embodiment of the invention will comprise the sequence of SEQ ID NO: 2, where Xaa is selected from a group that includes, but is not limited to, alanine, valine, leucine, tyrosine, and proline. Resulting preferred embodiments would include, Ala-SerP-SerP-Glu-Glu, Val-SerP-SerP-Glu-Glu, Leu-SerP-SerP-Glu-Glu, Tyr-SerP-SerP-Glu-Glu, and Pro-SerP-SerP-Glu-Glu.

In another embodiment of the invention, multiple copies of CPP comprising the mineral-binding target sequence are connected by links that are susceptible to enzymatic cleavage. Said links may be designed to be particularly susceptible to cleavage in the digestive tract. Another embodiment may comprise links designed to be particularly susceptible to cleavage in the mouth environment. In another embodiment, said links may be designed for cleavage in vitro, for example if necessary for large-scale CPP production.

The phosphopeptides may be from any source. In one preferred embodiment, the peptides are chemically synthesized by well-known methods using an amino acid synthesizer. In another preferred embodiment, the phosphoproteins and phosphopeptides are recombinantly produced using well-known methods such as those described in Sambrook et al., Molecular Cloning: A Laboratory Manual, Cold Spring Harbor Laboratory Press, Cold Spring Harbor, N.Y., 1988. If the peptides are recombinantly produced, they are then phosphorylated using a serine kinase, for example by the method of Chardot, et al., Nahrung. 42: 145-147 (1998).

The beta-casein (β-CN)of the present invention can be derived from and correspond to any native mammalian beta-casein wherein the first of the three native serine residues in the conserved sequence Ser-Ser-Ser-Glu-Glu has been deleted or replaced with another amino acid. Beta-caseins of human, bovine, goat, porcine, or other non-murine source may be used, either as whole proteins or protein fragments that include the sequence of SEQ ID NO: 2. These protein fragments are preferably from about 5, 6, 7, 8, 9, or 10 amino acids up to about 20, 30, 40, 50, 80, or 100 amino acids in length.

Variants of non-murine β-CN in which the first SerP contained within SEQ ID NO: 1 has been changed to any other amino acid besides serine or phosphoserine can be produced by well-known methods, including site-directed mutagenesis. The resulting mineral-binding domain having only two SerP residues binds calcium and other minerals with significantly lower affinity than the corresponding binding domain which contains three, four or five SerP residues (Table 1). This is desirable because the minerals can be more easily released within the body due to the lower binding affinity. Prior art using β-CN having the calcium binding amino acid sequence of SEQ ID NO: 1 have been directed to the highest binding affinity possible. In contrast, this invention uses the lower binding affinity of the target sequence of the human β-CN variant comprising SEQ ID NO: 2 to more readily release the minerals complexed thereto.

The β-CN variants and peptides derived therefrom having the Xaa-SerP-SerP-Glu-Glu sequence (SEQ ID NO: 2) can be complexed to any desired mineral prior to administration to a vertebrate, more preferably a bird or mammal, and most preferably a human.

Complexes, or aggregates, can be formed by mixing these peptides or proteins with a salt of the desired mineral in the presence of a suitable solvent, such as water. For example, the proteins or peptides may be complexed with calcium for use as a calcium supplement to increase calcium bioavailability and to treat or prevent diseases associated with calcium deficiencies. The calcium supplements of the invention may be administered to pre- or postmenopausal women prone to or afflicted with osteoporosis or osteomalacia, given to infants and children as a calcium supplement, and used for prevention of dental caries. Compositions including calcium phosphate are desirable for their ability to deliver both calcium and phosphate. Compositions can also include vitamin D and/or 1,25-Dihydroxy Vitamin D (1,25-Dihydroxycholecalciferol), or other components that enhance calcium absorption.

Of particular interest in the present invention are nutritional or prophylactic compositions that include carbamoyl phosphate or other carbamic acid derivatives. Carbamoyl phosphate has been shown to have a prophylactic and possible therapeutic effect on dental caries. It has now been discovered that carbamoyl phosphate and other carbamate compounds have a salutary effect on stabilization or growth of bone tissue and bone density.

Thus, the present invention contemplates administering carbamoyl phosphate, or carbamoyl phosphate in addition to CPP or other compounds of the present invention, in an amount that is effective to facilitate calcification of bone tissue, or that is sufficient to prevent or retard decalcification of bone tissue. Similarly, carbamoyl phosphate can be used with the CPP-related compounds of the present invention to inhibit calcium loss in teeth, or to facilitate calcification or recalcification of teeth, as well as to prevent or treat dental caries. An effective amount for any particular species can be readily determined through empirical means. For example, for a mammal (including a human), from 0.1 to 100 mmol/kg may advantageously be administered each day, more preferably from about 1 to about 10 mmol/kg. The carbamoyl compositions of the present invention preferably include any suitable source of dietary calcium (e.g., calcium citrate or calcium picolinate), and/or one or more other minerals in suitable dietary form (e.g., chromium, magnesium, iron, zinc, copper, selenium, manganese, and molybdenum). Such compositions may also optionally include CPP compounds. The carbamoyl compositions of the present invention may be formulated as food or food additive compositions, pills, elixirs, injectable formulations, or any of the other forms described herein in connection with CPP.

A high sucrose diet is associated with a decrease in bone specific gravity in growing rats (reflecting reduced calcium and phosphorous content). See J. Nutr. 128:1807-1810 (1998). This decrease in bone density is reversed or prevented by carbamoyl phosphate supplementation. Sprague-Dawley rats were fed a high sucrose diet for 5 weeks, supplemented with carbarnoyl phosphate at either 6.25 or 3.125 mmol/kg of diet. The specific gravities of femurs and tibias of both groups of rats receiving carbamoyl phosphate was significantly higher than rats on the same diet without carbamoyl phosphate, and were not significantly different from control rats receiving standard lab chow. Thus, carbamoyl phosphate modifies the effect of a high sucrose diet on bone growth.

Note that in addition to carbamoyl phosphate, other carbamate derivatives can also be used, including without limitation, carbamoyl aspartate. Other amino acid derivatives of carbamic acid are also within the scope of the invention, including derivatives formed from each of the 20 common amino acids in mammalian proteins, and more specifically each of the hydrophobic amino acids, each of the hydrophilic amino acids, each of the charged amino acids, each of the polar amino acids, as well as dipeptide, tripeptide, and larger derivatives of carbamic acid. Further within the scope of the invention are carbamate derivatives of each of the CPP-related compounds disclosed herein. For example, the 5-mer of SEQ ID NO: 2 can be further derivatized by condensation with carbamic acid, and carbamoyl compounds formed from longer peptides falling within the scope of the present invention are also contemplated. In each case, the compositions may optionally include Vitamin D, in an amount consistent with normal mammalian nutritional or supplementation with that vitamin. Note that the U.S. recommended daily allowance of Vitamin D is 400 IU, and individual daily dosages of, for example, 10 to 1000 IU within the scope of the present invention may be advantageous in facilitating or enhancing bone and tooth calcification. Equally preferably, the compositions may optionally include 1,25-Dihydroxy Vitamin D (1,25-Dihydroxycholecalciferol), alone or in combination with Vitamin D, in an amount consistent with normal mammalian nutritional or supplementation with that vitamin.

The invention also relates to methods of treating conditions relating to calcium loss from the body (especially from the bones), calcium deficiency, calcium malabsorption and other similar conditions. The phosphopeptide-calcium complexes and carbamoyl compositions of the invention can be used to treat any condition requiring increased calcium bioavailability. The complexes are also useful for individuals needing stimulation of bone growth, including those undergoing joint replacement, craniofacial surgery, bone grafts, fracture repair and the like. These complexes are also useful as calcium supplements in individuals who cannot consume dairy products as their main source of calcium, such as individuals with lactose intolerance and milk allergies.

The proteins and peptides of the invention can also be complexed with other minerals including magnesium, iron, zinc, copper, selenium, manganese, molybdenum, chromium and the like. Chromium is known to play a beneficial role in reduction of blood sugar levels in individuals with adult-onset (Type II) diabetes. However, chromium in most forms is poorly absorbed. Thus, the complex of chromium with the phosphopeptides and phosphoproteins of the invention provides a good, readily absorbable form of chromium. The proteins and peptides of the invention can be complexed to any mineral which is nutritionally beneficial.

In one preferred embodiment of the invention, the phosphopeptide-calcium complex is incorporated into dental preparations including toothpaste, mouthwash, chewing gums, dairy products and other foods using methods well known in the art, such as those described in U.S. Pat. No. 5,227,154, the entire contents of which are hereby incorporated by reference, or Reynolds, WO98/40406, similarly incorporated herein in its entirety, for use in prevention and/or treatment of dental caries or tooth decay. The use of chewing gums and foods containing the phosphopeptide-calcium complexes of the invention also provides calcium for other needs, such as increasing bone density. The phosphopeptide-calcium composition may comprise, for example, 0.05-50% by weight of the nutritional or prophylactic composition, and preferably 1.0-50%. These oral compositions may also include additional ingredients conventional to the preparation of these formulations. Dosages may be determined by one of ordinary skill in the art as appropriate for particular uses. Typically, the total dosage for an adult human for nutritional purposes (e.g., calcium supplementation) is equivalent to from 0.001% to about 5% of the daily intake of solid food per day, more preferably from about 0.01% to about 1%.

For oral administration, the compositions of the invention may be incorporated into a tablet, an aqueous or oral suspension, a dispersible powder or granule, a microbead, emulsion, hard or soft capsule, syrup, or elixir. Compositions may be prepared according to any method known in the art for the manufacture of pharmaceutically acceptable compositions and such compositions may contain one or more of the following agents: sweeteners, flavoring agents, coloring agents and preservatives. Tablets containing the active ingredient in admixture with non-toxic pharmaceutically acceptable excipients suitable for tablet manufacture are acceptable. “Pharmaceutically acceptable” means that the agent should be acceptable in the sense of being comparable with the other ingredients of the formulation, as well as non-injurious to the individual. These excipients include inert diluents such as calcium carbonate, sodium carbonate, lactose, calcium phosphate or sodium phosphate; granulating and disintegrating agents such as corn starch and alginic acid; binding agents such as starch, gelatin or acacia; and lubricating agents such as magnesium stearate, stearic acid or talc. Tablets may be uncoated or may be coated by known techniques to delay disintegration and absorption in the gastrointestinal tract and thereby provide a sustained action over a longer period of time. For example, a time delay material such as glyceryl monostearate or glyceryl stearate alone or with a wax may be employed.

In another preferred embodiment, tablets, capsules or microbeads are coated with an enteric coating which prevents dissolution in the acidic environment of the stomach. Instead, this coating dissolves in the small intestine at a more neutral pH. Such enteric coated compositions are described by Bauer et al., Coated Pharmaceutical Dosage Forms: Fundamentals, Manufacturing Techniques, Biopharmaceutical Aspects, Test Methods and Raw Materials, CRC Press, Washington, D.C., 1998.

Formations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Aqueous suspensions may contain the compositions of the invention in admixture with excipients for the manufacture of aqueous suspensions. Such excipients include suspending agents, dispersing or wetting agents, one or more preservatives, one or more coloring agents, one or more flavoring agents and one or more sweetening agents such as sucrose or saccharin.

Oil suspensions may be formulated by suspending the active ingredient in a vegetable oil, such as peanut oil, olive oil, sesame oil or coconut oil, or in a mineral oil such as liquid paraffin. The oil suspension may contain a thickening agent, such as beeswax, hard paraffin or cetyl alcohol. Sweetening agents, such as those set forth above, and flavoring agents may be added to provide a palatable oral preparation. These compositions may be preserved by an added antioxidant such as ascorbic acid. Dispersible powders and granules of the invention suitable for preparation of an aqueous suspension by the addition of water provide the ingredient in admixture with a dispersing or wetting agent, a suspending agent, and one or more preservatives. Additional excipients, for example sweetening, flavoring and coloring agents, may also be present.

Syrups and elixirs may be formulated with sweetening agents, such as glycerol, sorbitol or sucrose. Such formulations may also contain a demulcent, a preservative, a flavoring or a coloring agent.

It will be appreciated that although specific embodiments of the invention have been described herein for purposes of illustration, various modifications may be made without deviating from the spirit and scope of the invention. Accordingly, the invention is not limited except as by the appended claims.

What is claimed is:

1. An isolated recombinant or synthetic beta-casein (β-CN) variant or a peptide, comprising the amino acid sequence Xaa-SerP-SerP-Glu-Glu, wherein Xaa is an amino acid other than serine or phosphoserine.

2. A composition comprising an isolated non-murine beta-casein (β-CN) variant or a peptide, comprising the amino acid sequence Xaa-SerP-SerP-Glu-Glu, wherein Xaa is an amino acid other than serine or phosphoserine, in combination with a mineral.

3. The composition of claim 2, wherein said β-CN is recombinant.

4. The composition of claim 2, wherein said mineral is selected from calcium, inorganic phosphate, chromium, magnesium, iron, zinc, copper, selenium, manganese, and molybdenum.

5. The composition of claim 2, further comprising a carbamoyl compound.

6. The composition of claim 5, wherein the carbamoyl compound is carbamoyl phosphate or carbamoyl aspartate.

7. The composition of claim 2, wherein Xaa is selected from alanine, valine, leucine, tyrosine, and proline.

8. The composition of claim 2, further comprising Vitamin D or 1,25-Dihydroxy Vitamin D or a combination of Vitamin D and 1,25-Dihydroxy Vitamin D.

9. A method for delivering a mineral to an individual in need thereof, comprising administering to said individual an isolated beta-casein (β-CN) variant or a peptide, comprising the amino acid sequence Xaa-SerP-SerP-Glu-Glu, wherein Xaa is an amino acid other than serine or phosphoserine, in combination with said mineral.

10. The method of claim 9, wherein said mineral is selected from the group consisting of calcium, inorganic phosphate, chromium, magnesium, iron, zinc, copper, selenium, manganese, and molybdenum.

11. A method for delivering a mineral to an individual in need thereof, comprising administering to said individual an isolated peptide having between about 8 and 50 amino acids, comprising the amino acid sequence Xaa-SerP-SerP-Glu-Glu, wherein Xaa is an amino acid other than serine, phosphoserine, aspartate, or glutamate, in combination with said mineral.

12. The method of claim 11, wherein said mineral is selected from the group consisting of calcium, inorganic phosphate, chromium, magnesium, iron, zinc, copper, selenium, manganese, and molybdenum.

13. The method of claim 11, wherein said peptide is synthetic.

14. A dental formulation comprising an isolated recombinant or synthetic human beta-casein (β-CN) variant or peptide comprising the amino acid sequence Xaa-SerP-SerP-Glu-Glu, wherein Xaa is an amino acid other than serine or phosphoserine.

15. The dental formulation of claim 14, wherein said formulation is selected from the group consisting of a toothpaste, mouthwash and chewing gum.

16. A method for increasing bone mass or preventing or reducing bone loss, comprising administering to a vertebrate a synthetic or recombinant peptide or protein comprising the amino acid sequence Xaa-SerP-SerP-Glu-Glu, wherein Xaa is an amino acid other than serine or phosphoserine, in combination with calcium.

17. The method of claim 16, further comprising administering a carbamoyl compound to the vertebrate.

18. The method of claim 17, wherein the carbamoyl compound is carbamoyl phosphate or carbamoyl aspartate.

19. The method of claim 16, further comprising administering Vitamin D or 1,25-Dihydroxy Vitamin D or a combination of Vitamin D and 1,25-Dihydroxy Vitamin D to the vertebrate.

20. A method for increasing bone density or inhibiting loss of bone density, comprising administering to a vertebrate in need thereof an effective amount of a carbamate or carbamoyl compound.

21. The method of claim 20, wherein the carbamoyl compound is carbamoyl phosphate.

22. The method of claim 21, further comprising administering an effective dietary supplement of calcium to the vertebrate.

23. The method of claim 22, further comprising administering an effective amount of inorganic phosphate, chromium, magnesium, iron, zinc, copper, selenium, manganese, or molybdenum to the vertebrate.
